(19) United States
(12) Patent Application Publication
(10) Pub. No.: US 2003/0158129 A1
(43) Pub. Date: Aug. 21, 2003

(54) Vectors and methods for providing cells with additional nucleic acid material integrated in the genome of said cells

(76) Inventors: Ronald Hans Plasterk, Bussum (NL); Jan Christiaan Vos, Amsterdam (NL)

Correspondence Address: TRASK BRITT, P.O. BOX 2550, SALT LAKE CITY, UT, 84110, US

(21) Appl. No.: 09/731,289

(22) Filed: Dec. 6, 2000

Related U.S. Application Data
(63) Continuation of application No. 09/117,985, filed on Aug. 7, 1998, now abandoned.
Application No. 09/117,985 is a 371 of international application No. PCT/NL97/00040, filed on Feb. 7, 1997.

(30) Foreign Application Priority Data
Feb. 9, 1996 (EP) 96200298.6

(51) Int. Cl. A61K 48/00; C12N 15/861

(52) U.S. Cl. 514/44; 435/320.1

(57) ABSTRACT

The present invention provides elements for producing recombinant nucliec acid molecules and/or recombinant cells. The elements are capable of integrating desired nucleic acid material into other nucleic acid materials, specifically into the genome of a host cell. The elements are derived from or based an transposons, in particular from the Tc/Mariner superfamily. In particular the essential elements of Tc1 enabling excision and pasting of the desired nucleic acid material are provided, together with the relevant transposase activity in cis or in trans.

The present invention relates to the field of genetic engineering, in particular to the field of methods for making recombinant vectors containing nucleic acids of interest, methods for making recombinant cells, preferably expressing recombinant products, and/or production of nucleic acids of interest.

In a further embodiment the invention relates to transgenesis of animals, plants or other organisms of medical, scientific or economic interest. In yet another embodiment the invention relates to tools for mutagenesis as well as to tools and means for localization and/or identification of nucleic acid sequences of interest (such as genes) in the genome of a host.

Many ways of providing cells with additional nucleic acids are now known in the field. Many proteins have been expressed in many kinds of cells, ranging from prokaryotic bacteria and bacilli, via yeast and fungi to plant cells, insect cells an mammalian cells.

Often expression of proteins as discussed above has met with success, there are however, certain areas in the field of recombinant technology where, because of safety issues or technical problems, such as stability of the additional nucleic acid material introduced, success has not been easy to achieve. Also, in the application of genetic engineering where the product is not a protein, but for instance a nucleic acid of interest (such as DNA, RNA or an antisense construct), the same or similar problems have been encountered. It is in these areas particularly that the present invention finds its use.

One of the problems encountered in recombinant technology is that it is often desirable to have the additional nucleic acid material, which is introduced into a host cell, integrate in the genome of said host cell. Once the additional nucleic acid material is integrated in the genome its stability is much less an issue. Moreover, the integrated material is reiplidated together with the genome and thus will be present in the offspring of the recombinant cell as well. Vectors which are capable of providing for integration of additional nucleic acid material into a host cell are known, but they do suffer from some drawbacks which will be discussed below.

The present invention now provides a way in which a wide variety of vectors can be altered to integrate the desired additional nucleic acid in the genome of the host cell.

This means that vectors which did not (efficiently) integrate the desired additional nucleic acid material in the genome of the host cell, can now be provided with said ability.

One of the drawbacks of the integrating vectors of the prior art is that they are often not capable of transducing a host cell efficiently. Techniques such as electroporation and the like are then necessary for achieving transduction.

In some areas such treatments of cells may be unwanted or impossible. One such an area is for instance gene therapy. In such cases vectors which can efficiently transduce host cells on their own, or which can be packaged into recombinant viral particles and so infect host cells are often employed.

However, many of these vectors are then incapable of (efficient) integration of the desired additional nucleic acid material in the genome of the host. The present invention thus offers the best of both worlds in that it enables to prepare vectors capable of efficiently transducing and efficiently integrating desired additional nucleic acid material in a host cell. The inventions solves this problem in that the nucleic acid to be integrated into a host cell genome is provided within a functional transposon. Exogenous DNA has been introduced into the genome of a host using a transposon, however since transposons were, until the present invention thought to be rather species specific, the applicability of such a system was thought to be very limited. At best, it was shown that a transposon functional in one fruit fly could also be used to integrate DNA into the genome of another species of fruit fly (Loutheris et al., 1995). The present inventors have found that, at least for a certain class of transposons, it is possible to use these transposons across wide phylogenetic barriers, which allows for wide application of these transposons as integration means for DNA into a wide variety of host genomes.

The invention thus provides a vector for providing a cell of a certain genus with additional nucleic acid material integrated in its genome, whereby said vector comprises two transposase binding sites, whereby said transposase binding sites may be the same or different and are derived from a transposon found in another genus, each in close proximity to a cut site for said transposase, whereby said additional nucleic acid material is located between said two transposase binding sites. It is of course clear that it is highly preferred to be able to use transpcsons over even wider phylogenetic barriers. The present invention thus provides a transposon-based integration system with a wide applicability.

Clear advantages of this system are for instance that: using a transposon will often lead to a more efficient integration into the host cell genome: using a transposon gives complete control of the integrating sequence (the termini of the integrating element are known): in the embodiment where gene (or DNA) localization is desired it will be an advantage that a transposon can integrate anywhere in the genome (especially useful in so-called mutagenesis experiments aimed at gene function, or in “gene-trap” experiments directed at tracing genes of interesting expression patterns). For expression purposes it may be desirable to develop transposons with a more specific integration site in the genome, or to develop methods of integration which lead to a more specific integration site.

Preferably said transposase binding sites and said cut sites are based on the corresponding sites in transposons from the Tc1/mariner superfamily of transposons, more preferably they are based on Tc1-like transposons. A very useful set of transposon elements is the minimum set required by the Tc1 transposon i.e. comprising the terminal 26 basepairs of Tc1 and in close proximity the cut site (TA) for Tc1 transposase.

An important advantage of the transposons according to the invention is that they are self-sufficient. All that is needed is a functional transposase binding site, a transposase cut site and transposase activity functional for those sites. Transposase activity needed should be as limited as possible. The preferred class of transposons only needs a single transposase. A reason for the species-specificity of transposons reported until to date may be that transposons have been used that require host proteins in order to be able to jump. The host proteins may then be responsible for the species-specificity of for instance the transposons of bacterial origin, or the P-element. The class provided with the present invention needs no host-elements and has modest cis-trans requirements. Therefore these transposons are far more suitable for integrating nucleic acids of interest into the genome of a wide variety of hosts.

In the preferred embodiment where elements based on Tc1 are used such a transposon binding site seems to be at least the 26 terminal basepairs. Probably not all of these basepairs are essential for the function of this binding site. Some (conservative) changes in such a binding site may therefore be allowed. It seems that sequences of around 100 bp are most efficient as transposase recognition sites.

For the invention the requirement is that they are functional for the corresponding transposase-activity, which transposase of course may also be modified, mutated, shortened or lengthened when compared with the original transposase, as long as it has relevant transpsase activity. The transposase-activity may be encoded on the same vector as the other transposon elements, even as part of the transposon together with the desired additional nucleic acid material (in cis), but it may very suitably be provided to the cell to be transduced by another vector according to the invention or by another vector (in trans).

When the transposase-activity is provided in trans and is preferably encoded by a sequence under control of an inducable and/or repressible promoter, “jumping” of the transposon can be switched on and off. Once integration into the host genome has taken place this may be very useful.

A vector for the purposes of this invention is any nucleic acid vehicle which is capable of carrying the desired additional nucleic acid material and preferably capable of transduction of host cells and/or replication.

The desired additional nucleic acid material may be encoding a protein and thus comprise a gene and regulatory elements for expression. Proteins to be expressed will depend on the purpose of the transduction. Many useful proteins for may applications have been identified as good candidates for recombinant expression. All these may be expressed using the present invention.

Also, the additional nucleic acid material (DNA) may be transcribed (once transduced) into RNA molecules blocking transcription or translation of host cell (or viral) nucleic acids. Such additional nucleic acid material may for instance be an antisense RNA molecule.

Use of the invention in the field of gene therapy is particularly advantageous, as may be clear from the above. Viral vectors often contemplated for gene therapy (such as adenovirus, retrovirus or adenoassociated virus) which are not capable of efficiently integrating the desired nucleic acid material into the genome of the infected cell may now be provided with such capability. Other vectors not based on a virus, but provided with a means to deliver them to a target cell population can of course also be advantageously provided with an integration system according to the invention. Particularly the use of liposomes or polymers as a targetting system is contemplated.

As stated earlier the cell's progeny will also have that capability. Thus, when stemcells are produced according to the invention, this will lead to very efficient gene therapy regimes. The vectors (or the integration system) of the invention can of course also be used to produce transgenic animals, plants or other organisms of scientific, economic or medical interest. Useful applications for transgenesis are well known in the art.

Thus methods using the vectors according to the invention to transduce cells or animals, plants, etc. are also disclosed and part of the invention, as are cells, animals or plants, etc. obtainable by such methods. Use of the invention in mutagenesis studies and the like is another preferred embodiment, as discussed hereinbefore. The invention will now be explained in more detail using Tc1 as a non-limitative example.

### DETAILED DESCRIPTION

Tc1 belongs to the Tc1/mariner superfamily of transposons found in nematodes, arthropods and chordates (Henikoff 1992; Raddice et al. 1994; Robertson 1995; Plasterk 1995). Both vertical and horizontal transfer have contributed to the spread of these elements throughout the animal kingdom (Robertson 1993; Radice et al. 1994; Robertson and Lampe 1995). The widespread occurrence of the Tc1/mariner family of transposons can be taken as an idication for the absence of species-specific host factors which limit the transfer between different species. Therefore, Tc1/mariner elements are attractive candidates for the development of gene delivery vectors.

Tc1-like elements are close to 1.7 kb in length, have short inverted terminal repeats flanking a transposase gene and have the conserved sequence CAGT at their termini, flanked by TA representing the target site which is duplicated upon integration (Van Luenen et al. 1994). The element-encoded proteins share a homologous catalytic domain with bacterial transposases and retroviral integrases (Doak et al. 1994). Tc1 from C. elegans is a 1612 bp long transposon which has 54 bp inverted repeats flanking a gene encoding a 343 amino acid transposase (Emmons et al. 1983; Rosenzweig et al. 1983; Vos et al. 1993), that binds to the inverted repeats (Vos et al. 1993; Vos and Plasterk 1994). The conserved hexanucleotide sequence, TACAGT, at the extreme termini of the element is not part of the transposase binding site, but is thought to play a role in catalysis of the transposition reaction (Vos and Plasterk 1994).

Here, we describe in vitro excision and transposition of Tc1 using an extract prapared from transgenic nematodes. The minimal cis-requirements for transposition are defined and the target site choice in vitro is compared with that in vivo. Furthermore, we demonstrate that recombinant transposase purified from E. coli is capable of supporting transposition, showing that no other factors are essential for Tc1 transpostion in vitro.

Results

Transposition of Tc1 in vitro

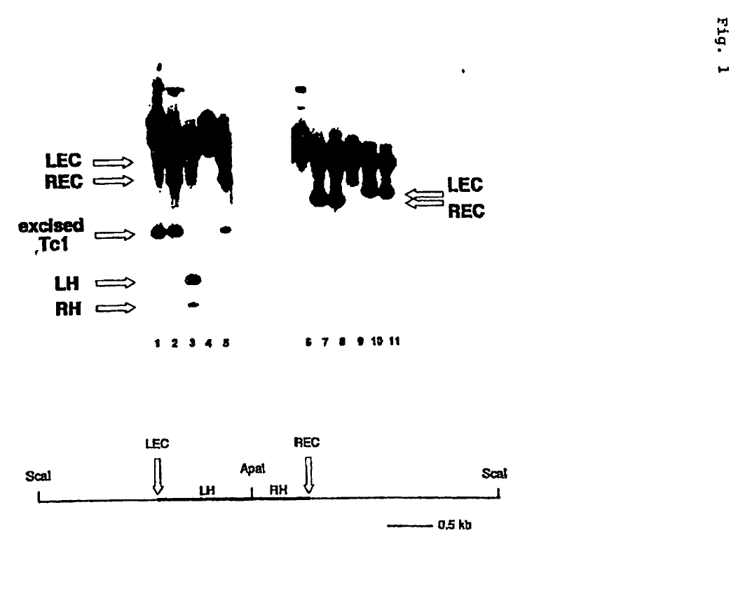

We generated a transgenic worm with the Tc1 transposase gene under the control of a heat shock promoter. This allowed the preparation of a nuclear extract with elevated levels of transposase, which proved to be highly important to detect activity. The extract was incubated with a plasmid containing a Tc1 element. Excision was studied in a physical assay. Southern blot analysis of reaction products shows the appearance of excised Tc1 elements (FIG. 1, lane 1). Furthermore, cleavage at either the left or the right end of Tc1 is detected when the products are digested with Scal within the plasmid backbone prior to electrophoresis (lane 2). Cleavage may require a divalent cation (Mg²⁺ or Mn²⁺) and is stimulated by the presence of ethylene glycol or 5% DMSO (data not shown). The efficiency of cleavage at a single end of the transposon is not decreased if the substrate is linear (compare lanes 2 and 5). Also, deletion of either end of the transposon does not abolish cleavage at the remaining end (lanes 6 and 11), which suggests that cleavage does not require interaction between the two ends. In contrast to single end cleavage, excision of the complete element is reduced about 2-fold when the substrate is linear, suggesting that coordinated cutting at both ends is stimulated by supercoiling of the substrate. The majority of complete excision products observed with a linear substrate can be explained by non-coordinated cleavages at both ends.

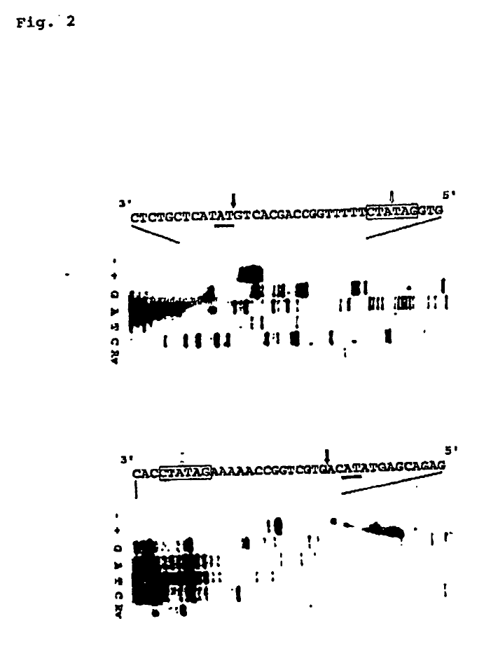

To determine the positions of the double strand cleavages at the nucleotide level, a PCR based primer extension was performed using end-labeled oligonucleotides specific for each strand (FIG. 2). The 5′ cut is 2 bp within the transposon, whereas the 3′ cut maps to the end of the transposon, as based on the largest observed PCR product. This confirms the model based on in vivo studies of the related C. elegans transposon Tc3, for which it was shown that excision results in a 2 bp staggered 3′ overhang (Van Luenen et al. 1994). The complete excision of Tc1 shows that transposition occurs via a cut-and-paste process, a result consistent with genetic data on double strand break repair of the donor DNA molecule upon Tc1 excision (Plasterk 1991).

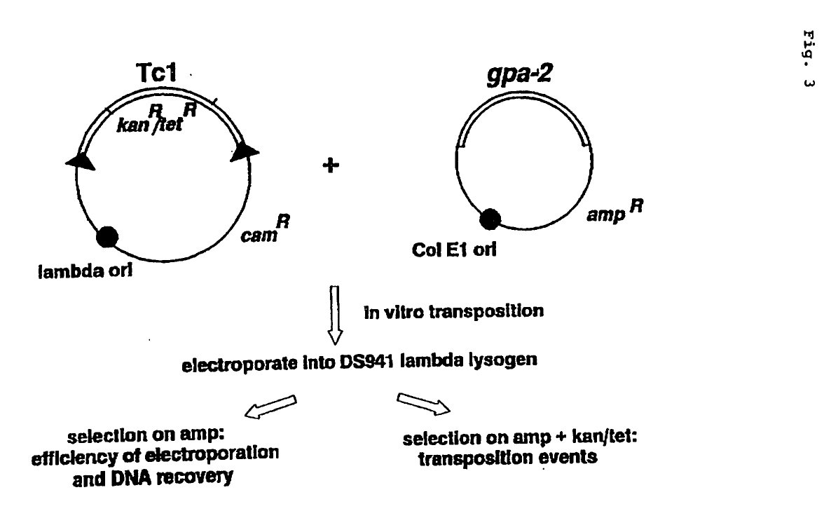

We devised a sensitive assay to detect integration events. We selected for jumping of a transposon-borne antibiotic resistance gene from a supercoiled donor plasmid to a target plasmid in a genetic assay (FIG. 3). Electroporation of reaction products into the appropriate E. coli strain resulted in the detection of many transposition events (Table 1). Extracts prepared from non-transgenic N2 worms or from the so-called high hopper strain, TR679 (Collins et al. 1987), which has a high frequencey of germline transposition, do not generate a detectable level of transposition products in this assey. Linerization of the donor plasmid resulted in an approximately 20-fold reduced efficiency of transposition. Transpositon requires two inverted repeat sequences, because no integrations were obtained upon deletion of one transposon end. Furthermore, the addition of ATP, GTP or dNTPs does not increase the level of transposition (data not shown), which indicates that the process is neutral in energy-consumption and independent of a cofactor. About 90 independent in vitro Tc1 integrations were analyzed by sequencing and found at TA dinucleotides, which had been duplicated in the process. Two odd integration events were detected, where Tc1 had integrated in the sequence TTG or CCT. In both cases, we found a 3 bp target site duplication.

Target Site Choice

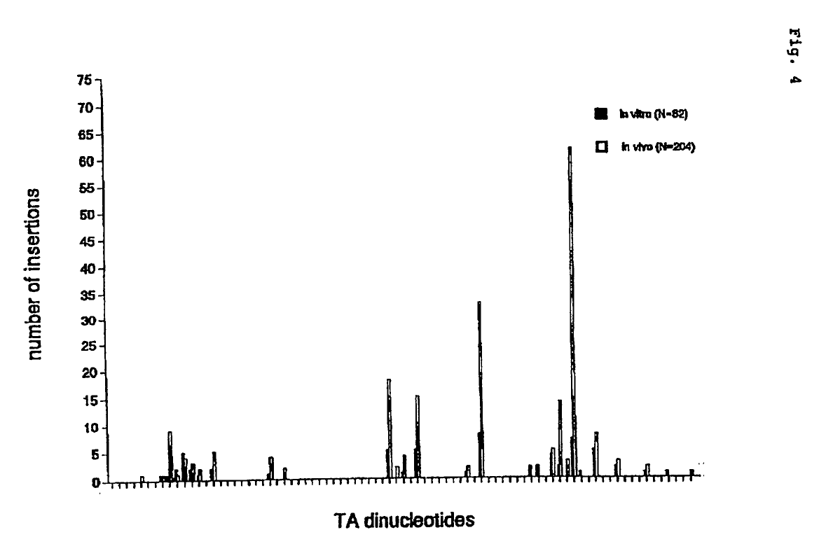

Previously, several hundreds of in vivo Tc1 and Tc3 integrations in a 1 kb region of the gpa-2 gene have been analyzed (Van Luenen and Plasterk 1994). This showed the selective use of a limited set of TA dinucleotides as targets of integration, with a striking difference in preference between Tc1 and Tc3. To investigate whether the chromatin structure played a role in the choice of integration sites, we determined the pattern of integrations into naked DNA in vitro, using the same target region previously assayed in vivo. Therefore, we included the gpa-2 region in our target plasmid. It is apparent that the same overall pattern of integration is seen (FIG. 4). Hot sites in vivo appear to be hot in vitro and old sites in vitro are also cold in vivo. This indicates that, at least in this region of the qenome, the genome, the chromatin structure or the transcriptional status of the DNA in vivo is not the major determinant of target choice.

Transposition by Recombinant Transposase

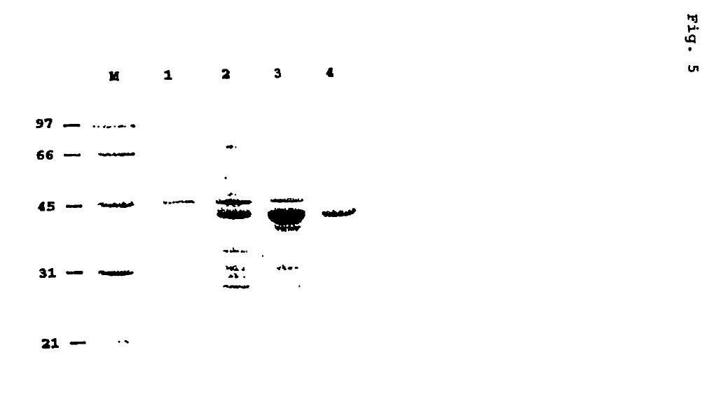

The nematode is not a convenient source of protein for an extensive purification of transposase. Therefore, we expressed the protein in a heterologous system. Both expression using Baculovirus and Sf9 cells (data not shown) or expression in E. coli yielded transposase capable of supporting Tc1 transposition. Recombinant transposase was purified from inclusion bodies to near homogeneity (FIG. 5). Table 1 shows the frequency of transposition when comparable amounts of transposase were used for both the worm extract and the purified protein. Sequence analysis of 9 independent integrations in case of the recombinant protein showed that transposition into TA target sequences that were duplicated, from which it can be concluded that bona fide transposition had occurred. Therefore, we conclude that Tc1 transposase is the only protein required for Tc1 transposition. The difference in efficiency between nematode derived and bacterial transposase needs further studies. It could reflect a folding problem of the bacterial transposase, which was denatured and refolded during the purification procedure, or the stimulatory role of host factors present in the nematode extract.

Minimal Cis-Requirements

We investigated the possibility that the terminal 26 bp of Tc1 which constitute a full transposase binding site, flanked by the TA target site, are sufficient to form an artificial transposon. An element consisting of only these Tc1-specific sequences is still able to transpose in vitro, albelt at a lower frequency (Table 1). We sequenced several integrations and found them to be correct.

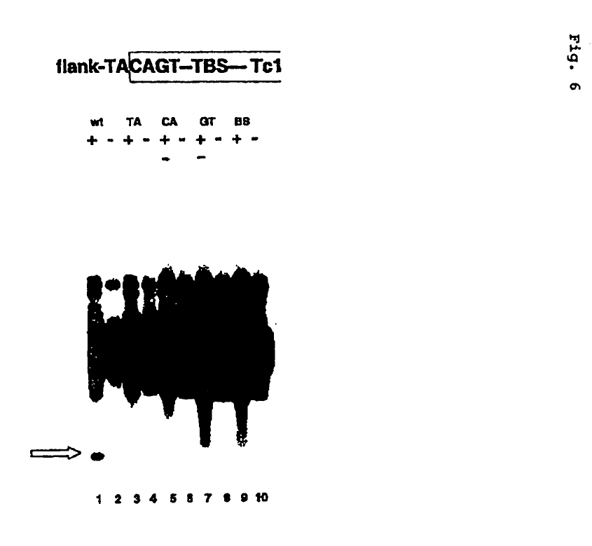

Furthermore, we investigated the importance of the conserved hexanucleotide sequence TACAGT. Mutations were introduced at one of the ends of a mini-Tc1 which contains only the terminal 26 bp as well as the flanking TA dinucleotide. Whereas excision of the element with 2 wild-type ends is easily detected in a physical assay, mutation of the transposase binding site, the flanking TA sequence of the termini, resulted in the inability of the element to excise (FIG. 6). Double strand cleavage at the wild type end was not affected by mutation at the other transposon end. Analysis of cleavage by PCR based primer extension revealed that, for the CA to TG mutation only, single stranded breaks at the 5′ end of the transposon had occurred (data not shown).

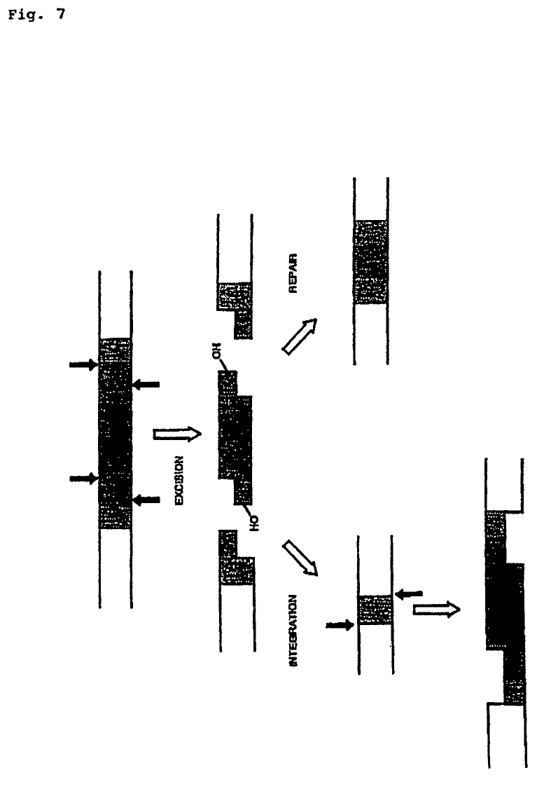

We have developed a cell-free Tc1 transposition system. Excision occcurs by double strand breaks at the transposon ends resulting in 2 bp staggered 3′ overhangs. A cut-and-paste mechanisme of transposition appears to apply for Tc1 (FIG. 7). This mechanism was already proposed on the basis of genetic data (Plasterk 1991) as well as the analysis of in vivo transposition products (Van Luenen et al. 1994). Nonreplicative transposition is shared with the bacterial transposons Tn7 (Bainton et al. 1991, 1993) and Tn10 (Bender and Kleckner 1986) as well as the Drosophila P element (Kaufman and Rio 1992). In contrast, the Mu and Tn3 transposable elements transpose via a replicative mechanism (Grindley and Sherratt 1978; Shapiro 1979; Mizuuchi 1992). Tc1 transposition appears to be independent of addition of a nucleotide cofactor, whereas P elements use GTP (Kaufman and Rio 1992) and Tn7 uses ATP as cofactor (Bainton et al. 1993).

A striking feature of the Tc1/mariner family is the absolute use of a TA dinucleotide as target site. An extensive study of target site choice in vivo had revealed the usage of only a subset of the available TA dinucleotides and a marked difference in target choice between the two related transposons Tc1 and Tc3 in C elegans (Van Luenen and Plasterk 1994). We find the same overall integration pattern in vitro as had been observed in vivo. This suggests that the chromosomal context of the DNA does not affect target choice, at least in the region of the genome analyzed. Therefore, we favor the idea that the transposition complex primarily selects its target site on the basis of the primary DNA sequence flanking the TA, although a strong consensus sequence could not be identified (Van Luenen and Plasterk 1994). A clear influence of the chromatin structure has been demonstrated for retroviral integrations (Pryciak and Varmus 1992; Müller and Varmus 1994). These studies showed a preference for regions within nucleosomal DNA, probably due to the bending of the DNA. We cannot exclude that DNA binding proteins can affect regional preferences for Tc1 integration. Because nothing is known about the chromosomal organization of the gpa-2 gene, it will be of interest to compare integration sites using reconsituted nucleosomal DNA in vitro.

Transposition in vitro requres the extreme termini of the transposon containing the transposase binding site and the conserved hexanucleotide sequence, which is important for excision. We observe a decrease in transposition efficiency between transposition of a full-length transposon and the Tc1 element with only 26 bp terminal inverted repeats, which suggests that additional sequences can contribute to transposition efficiency. We have no indications for additional transposase binding sites, but perhaps small basic proteins like high mobility group proteins (Grosschedl et al. 1994) may bind and stimulate transposition. Alternatively, unique A-T-rich sequences found at the transposon ends may add a helping bend to the DNA. The conserved hexanucleotide sequence at the extreme termini of the transposon are shown to be important at least for the cleavage step. The 5′ end single strand cleavage seen for one of the mutations (CA to TG) is perhaps an indication for a specific order of single strand cleavages, i.e. first the non-transferred strand, which would be the opposite of what has been reported for Tc10 (Bolland and Kleckner 1995).

Transposase purified from E. coli to near homogeneity is able to execute jumping of Tc1, which indicates that transposase is the only protein required for excision and integration of Tc1. The higher efficiency obtained with the nematode extract suggests that host factors may enhance the frequency of the reaction. It has for instance been shown that the mammalian proteins HMG1 and HMG2 can stimulate prokaryotic recombinations (Paull et al 1993). The independence of species-specific factors might be the explanation why members of the Tc1/mariner family are dispersed over so many different phyla, possibly by means of horizontal transfer (Robertson and Lanpe 1995). This is in contrast to P elements which are restricted to Drosophila species. Transposition of P elements in other species has not been observed (Rio et al. 1988). A possible candidate for a species-specific host factor in P transposition is the inverted repeat binding protein, IRBP (Beall et al. 1994). The simple cis- and trans-requirements for Tc1 transposition in vitro shows that this transposable element will be a good vector for gene delivery in a wide variety of animals. Using a marker gene, it has been shown that the Tc3 transposase can catalyze transposition of a recombinant Tc3 element in mammalian cells in culture.

### Materials and Methods

Plasmid Constructions

pRP466 contains a Tc1 element with 0.4 kb flanking sequences derived from plM40 (Mori 1988) cloned as a BamHI-XbaI fragment into pUC19, pRP467 and pRP468 are derivatives of pRP466 in which either a ClaI-Asp718 or PstI-ApaI fragment is deleted. pRP472 is a pACB104 (Boyd and Sherratt 1995) derivative which contains Tc1 with the AvaI-HindIII fragment of pBR322 inserted between the ClaI and ApaI sites. Cloning of the XbaI-BamHI fragment of pRP466 with the HindII Kan^{R}-cassette of pUC4K (Pharmacia) between the XhoI-sites into pACB104 resulted in pRP490. pRP491 is comparable to pRP490; all the internal Tc1 sequences have been replaced except the terminal 26 bp.

Transgenesis of C. elegans

A transgenic Bristol line was obtained after microinjection (Mello et al 1991) of 150 μg/ml pRP469 and 5 μg/ml pRP465 (Vos et al. 1993), 50 μg/ml pRF4 (Kramer et al. 1990) in strain CB1392 (nuc-1 9e1392)). A stable line, NL818(pkls221), was generated by X-ray irradiation (Way et al. 1991).

Extract Preparation

Stable line NL818 was grown in liquid culture at 18° C. and heat shocked for 3 hours at 33° C. to induce transposase expression. After 2 hours of further growth at 18° C., nuclear extracts were prepared as described (Vos et al. 1993) with differences in the buffers. NIB: 25 mM Tris pH 7.5, 20 mM KCl, 0.5 M sucrose, 0.5 mM EDTA, 5 mM β-mercaptoethanol, 0.1 mM PMSF, NEB: 25 mM Tris pH 7.5, 0.1 mM EDTA, 500 mM NaCl, 15% glycerol, 0.25% Tween-20, 0.1 mM PMSF, 1 mM DTT. Nuclear extract contains 2.5 mg/ml protein; concentration of Tc1A is about 10 μg/ml.

Recombinant Transposase Expression and Prurification

E. coli strain BL21 pLysS was transformed with pRP470 containing the Tc1 transposase gene under the control of a T7 promoter (Vos et al. 1993), grown in 2×YT medium and induced at an OD of 0.6 at 600 nm with 0.5 mM IPTG for 3 hours at 37° C. Inclusion bodies were purified as described by Nagai and Thogersen (1978). Inclusion bodies were dissolved in 8 M urea, 20 mM Na-phosphate pH 6.0 and loaded on a CM cellulose CL-6B column (Pharmacia). The protein was eluted with a linear gradient from 0 to 500 mM NaCl. The transposase containing fraction was loaded on a Sephacryl S400 HR gel filtration column equilibrated in 6 M guanidiumhydrochloride, 50 MM Tris pH 8.0. Transposase fractions were dialysed against 8 M urea, 50 mM Tris pH 8.0, 1 mM DTT. The protein was loaded on a S Sepharose FF column and eluted with 500 mM NaCl in the same buffer. All steps were performed at room temperature. The protein was renatured by a 100×dilution into ice-cold buffer 50 mM Tris pH 8.0, 100 mM NaCl, 5 mM DTT 5 mM MgCl₂. After 30 minutes, insoluble protein was removed by centrifugation for 15 minutes in an Eppendorf centrifuge. Transposase concentration was 200 μg/ml and estimated to be more than 90% pure.

In Vitro Transposition Reactions

Standard reaction conditions: 25 mM Tris pH 8.0, 25 mM NaCl, 1 mM DTT, 10% ethylene glycol, 5 mM MgCl₂ (or 2.5 mM EDTA), 4 mM spermidine, 0.05 μg/μl BSA. 200 ng of donor plasmid was preincubated with 2.5 μl worm extract or 0.25 μl of purified protein for 5 minutes on ice before addition of 2.5 μg target DNA in a total volume of 50 μl. Incubation was for 1 hour at 30° C. Reactions were stopped by addition of 5.5 μl of 250 mM Tris pH 8.0, 50 mM EDTA, 5% SDS, 2 mg/ml proteinase K. After 1 hour at 37° C., the DNA was precipitated and resuspended in 50 μl water.

Mapping of In Vitro Cleavage Sites

Linear PCR amplicifation was in 20 μl using 5 μl template and 0.5 pmol primer for 20 cycles; 1′ at 94° C., 1′ at 60° C., 1′ at 72° C., essentially as described (Craxton 1991). Sequence primers: BIGR=5′AGATTTCCACTTATATCATGTTTTATGTTTTGC, R2 (Van Luenen and Plasterk 1994).

Genetic Assay

Electrocompetent DS941 lambda lysogen (Flinn et al. 1989) bacteria were prepared and used as described (Zabarovsky and Winberg 1990). The donor plasmid contains a lambda origin of replication and can not replicate in the DS941 lambda lysogen; the target plasmid has Col E1 origin of replication. One to 5 μl of DNA was used per electrophoration and 5% of the bacteria were, after dilution, plated on ampicillin. The remaining bacteria were plated on double selection. This yielded, depending on the efficiency up to 200 transformants.

### References

Bainton, R. J., P. Gamas, and N. L. Craig 1991 Tn7 transposition in vitro proceeds through an excised transposon intermediate generated by staggered breaks in DdNA. Cell 65: 805-816.

Bainton R. J., K. M. kubo, J. n. Feng, and N. L. Craig, 1993.Tn7 transposition: target DNA recognition is mediated by multiple Tn7-encoded proteins in a purified in vitro system. Cell 72: 931-943.

Beall, E. L., A. Admon, and D. C. Rio, 1994. A Drosophila protein homologous to the human p70 Ku autoimmune antigen interacts with the P transposable element inverted repeats. Proc.Natl.Acad.Sci. USA 91: 12681-12685.

Bender, J. and N. Kleckner, 1986. Genetic evidence that Tn10 transposes by a nonreplicative mechanism. Cell 45: 801-815.

Bolland, S. and N. Kleckner, 1995. The two single-strand cleavages at each end of Tn10 occur in a specific order during transposition. Proc.Natl.Acad.Sci. USA 92: 7814-7818.

Boyd, A. /C. and D. J. Sherratt, 1995. The pCLIP plasmids:versatile cloning vectors based on the bacteriophage lambda origin of replication. Gene 153: 57-62.

Clar, J. M. 1988. Novel non-templated nucleotide addition catalyzed by procaryotic and eukaryotic DNA polymerases. Nucleic Acids Res. 16: 9677-9686.

Collins, J., B. Saari, and P. Anderson, 1987. Activation of a transposable element in the germ line but not the soma of Caenorhabditis elegans. Nature 328: 726-728.

Craxton, M, 1991. Linear amplification sequencing, a powerful method for sequencing DNA. METHODS: A companion to Meth. Enzymol. 3: 20-26.

Doak, T. G., F. P. Doerder, C. L. Jahn, and G. Herrick, 1994. A proposed superfamily of transposase-related genes: new members in transposon-like elements of ciliated protzoa and a common “D35E” motif. Proc.Natl.Acad.Sci. USA 91: 942-946.

Emmons, S. W., L. Yesner, K.-s. Ruan, and D. Katzenberg, 1983. Evidence for a transposon in Caenorhabditis elegans. Cell 32: 55-65.

Flinn, H. L., M. Burke, C. J. Stirling, and D. J Sherratt, 1989. Use of gene replacement to construct Escherichia coli strains carrying mutations in 2 genes required for stability of multicopy plasmids. J. Bacteriol. 171: 2241-2243.

Grindley, N. D. F. and D. J. Sherratt, 1978.Sequence analysis of IS1 insertion sites: models for transposition. Cold Spring Harbor Symp. Quant. Biol. 45: 125-133.

Grosschedl, R., K. Giese, and J. Pagel, 1994. HMG domain proteins: architectural elements in the assembly of nucleoprotein structures. Trends Genet. 10: 94-100.

Henikoff, S, 1992. Detection of Caenorhabditis transposon homologs in diverse organism. New Biol.: 382-388.

Kaufman, P. D., and D. C. Rio, 1992. P element transposition in vitro proceeds by a cut-and-paste mechanism and uses GTP as cofactor. Cell 69: 27-39.

Kramer, J. M., R. P. French, E. Park, and J. J. Johnson, 1990. The Caenorhabditis elegans rol-6 gene, which interacts with the sqt-1 collagen gene to determine organismal morphology encodes a collagen, Moll.Cel. Biol. 10: 2081-2089.

Loukeris, T. G., Livadaras, I., Arca, B., Zabalou, S., Savakis, C., 1995. Gene transfer into the medfly, Ceratitis capitata, with a Drosophila hydei transposable element. Science, 270(5244): 2002-5

Mello, C. C., J. M. Kramer, D. Stinchcomb, and V. Ambros, 1991. Efficient gene transfer in C. elegans: extrachromosomal maintenance and integration of transforming sequences. EMBO J. 10: 3959-3970.

Mizuuchi, K. 1992. Transpositional recombination: mechanistic insights from studies of Mu and other elements. Annu. Rev. Biochem.61: 1011-1051.

Mori, I. 1988. “Analysis of germline transposition and excision of Tc1 transposable elements in Caenorhabditis elegans”. Ph. D. thesis. Washington University, St. Louis, Mo.

Müller, H. -P. and H. E. Varmus, 1994. DNA bending create favored sites for retroviral integration: an explanation for preferred insertion sites in nucleosomes EMBO J. 13: 4704-4714.

Nagai, K. and H. C. Thogersen, 1987; Synthesis and sequence-specific proteolysis of hybrid proteins produced in Escherichia coli. Methods Enzymol. 153: 461-481

Paull, T. T., M. J. Haykinson, and R. C. Johnson, 1993. The nonspecific DNA-binding and -bending proteins HMG1 and HMG2 promote the assembly of complex nucleoprotein structures. Genes & Dev. 7: 1521-1534.

Plasterk, R. H. A. 1991. The origin of footprints of the Tc1 transposon of Caenorhabditis elegans, EMBO J. 10: 1919-1925.

Plasterk, R. H. A., 1995. The Tc1/mariner transposon family. In Transposable Elements (eds. H. Seadler and A. Gierl), Springer Verlag, Heidelberg.

Pryciak, P. M. and H. E. Varmus, 1992. Nucleosomes, DNA-binding proteins, and DNA sequence modulate retroviral integration target site selection. Cell 69: 769-780.

Radice, A. D., B. Bugaj, D. H. A. Fitch, and S. W. Emmons, 1994. Widespread occurrence of the Tc1 transposon family: Tc1-like transposons from teleost fish. Mol. Gen. Genet. 244: 606-612.

Rio, D. C., G. Barnes, F. A. Laski, J. Rine, and G. M. Rubin, 1988. Evidence for Drosophila P element transposase activity in mamalian cells and yeast. J. Mol. Biol. 200: 411-415.

Robertson, H. M. 1995. The Tc1 -mariner superfamily of transposons in animals. J.Insect. Physiol. 41: 99-105.

Robertson, H. M. 1993. The mariner transposable element is widespread in insects. Natrure 362: 241-245.

Robertson, H. M. and D. J. Lmape, 1995. Recent horizontal transfer of a mariner transposable element among and between Diptera and Neuroptera. Mol. Biol. Evol. 12: 850-862.

Rosenzweig, B., L. W. Liao, and D. Hirsch, 1983. Sequence of the C. elegans transposable element Tc1. Nucleic Acids Res. 12: 4201-4209.

Shapiro, J. A, 1979. Molecular model for the transposition and replication of bacteriophage Mu and other transposable elements. Proc. Natl. Acad. Sci. USA 76: 1933-1937.

Van Luenen, H. G. A. M. and R. H. A. Plasterk, 1994. Target site choice of the related transposable elements Tc1 and Tc3 of Caenorhabditis eleqans, Nucleic Acids Res. 22; 262-269.

Van Luenen, H. G. A. M. van Luenen, and R. H. A. Palasterk, 1993. CharActerization of the Caenorhabditis elegans Tc1 transposase in vivo and in vitro. Genes & Dev. 7: 1244-1253.

Vos, J. C. and R. H. A. Plasterk, 1994. Tc1 transposase of Caenorhabditis elegans is an endonuclease with a bipartite DNA binding domain. EMBO J. 13: 6125-6132.

Way, J. C., L. Wang, J. -O. Run, and A. Wang, 1991. The mec-3 gene contains cis-acting elements mediating positive and negative regulation in cells produced by asymmetric cell division in Caenorhabditis elegans. Genes & Dev. 5: 2199-2211.

Zabarosky, E. R. and G. Winberg, 1990. High efficiency electrophoration of ligated DNA into bacteria. Nucleic Acids Res. 18: 5912.

Legend

FIG. 1. Southern blot analysis of in vitro Tc1 transposition reaction products. Products of in vitro transposition reactions were separated on a 1% agarose gel, transferred to nitrocellulose and probed with radiolabeled Tc1. Standard reactions contained Mgcl₂ (lanes 1 to 3 and 5 to 11) or EDTA (lane 4). Products were digested with ScaI in vector DNA (lane 2, 7, 10) or ApaI in Tc1 DNA (lane 3) prior to electrophoresis. Lanes 5, 8 and 11 show reaction products when the substrate is lenearized with ScaI prior to in vitro cleavage. Lanes 1 to 5 show reaction products using pRP466 as substrate which carries a complete Tc1 element (see FIG. 4). Lanes 6 to 8 use pRP467 as substrate which has a deleted left end of Tc1, whereas lanes 9 to 11 show pRP4678 as substrate, which has the right end of Tc1 deleted. REC and LEC stands for Right and Left End Cleavage, respectively. RH and LH indicate the positions of the Right and Left Half of Tc1. A schematic of ScaI-linearized pRP466 is shown at the bottom of the figure.

FIG. 2. Mapping of the in vitro cleavage sites at the nucleotide level. A PCR based primer extension was performed on reaction products obtained in the presence of Mgcl₂ (+) or EDTRA(−) using pRP466 as donor. A control reaction was performed with pRP466 digested with EcoRV (RV lanes) to demonstrate the addition of one extra nucleotide at the end of the PCR product by Taq polymerase (see Clark 1988). Products were analyzed on a sequencing gel. Sequence reactions (GATC) were loaded as markers. PCR was with primer R2 (right panel) or primer BIGR (left panel). The relevant sequence is indicated with the EcoRV site boxed and the TA target site underlined. The cleavage sites are shown by arrows. Identical results were obtained when the positions of cleavage at the other transposon end was determined.

FIG. 3. Schematic representation of the genetic transposition assay. The donor plasmid, a pACB104 derivative (Boyd and Sherratt 1995) with a lambda origin of replication, contains a Tc1 element carrying an antibiotic resistance gene. The target plasmid, pRP475, carries a 1,4 kb hindII gpa-2 fragment and a Col, E1 origin of replication (pSP72, Promega). Reaction products were electroporated into a lambda lysogen. E. coli strain to counterselect against the donor. Integration events were selected on double antibiotics.

FIG. 4. Target site selection. Comparison of the distribution of in vitro (black bars) and in vivo (open bars) Tc1 insertions. pRP472 as donor and pRP475 as target were used in standard in vitro transposition reactions using C. elegans extract. Every mark on the X-axis represents a TA dinucleotide in the gpa-2 fragment as described in detail elsewhere (Van Luenen and Plasterk 1994).

FIG. 5. Purification of Tc1 transposase from E. coli. Analysis of transposase purified from inclusion bodies on a 12% SDS-polyacrylamide gel. Lane M: molecular weight markers (indicated in KDa); lane 1: bacterial lysate before induction; lane 2: bacterial lysate after induction: lane 3: purified inclusion bodies; lane 4: purified transposase after refolding.

FIG. 6. Mutations at the extreme termini of Tc1 affect excision. In vitro reaction products were obtained using C. elegans extract in the presence of Mgcl₂ (+) or EDTE (−) using as donor pRP480 (wt), pRP481 (TA), pRP482 (CA), pRP483 (GT) or pRP484 (BS), as indicated at the top. Products were separated on a 1% agarose gel, transferred to nitrocellulose and probed with radiolabeled Kan^{R}-gene fragment. The donor plasmids contain 28-mers cloned into the SmaI-site (wt sequence) and the HindII-site (wt or mutant sequence) of pUC19 with the Kan^{R}-cassette of pUC4K in between. TA was mutated to CG, CA to TG and GT to AC, respectively. In the transposase binding site mutation the BalI and EcoRV sites are mutated to TCCCA and GGGCCC, respectively (see Vos and Plasterk 1994).

FIG. 7. Model for Tc1 transposition. A model for non-replicative Tc1 transposition showing the excised, linear element with a 2 bp 3′ staggered overhang. Integration results in a duplication of the TA target-site. Repair of the double strand break leads to the generation of characteristic footprints (see also Van Luenen et al. 1994).

Table 1. Transposition frequeneies. In vitro Tc1 transposition reactions were carried out with supercoiled (SC) or linear donor plasmids and with protein sources as indicated and the ratios of amp^{R}-kan^{R} to amp^{R} colonies (*10⁸) are shown for two independent experiments. No integration products were recovered when reactions were performed in the presence of EDTA.

TABLE 1

| Donor | source | exp. 1 | exp. 2 |
|---|---|---|---|
| pRP490, Sc | C. elegans | 21 | 22 |
| pRP490, linear | C. elegans | 0.5 | 1.0 |
| pRP490, SC | C. elegans | 3.7 | 1.6 |
| pRP490, SC | E. coli | 3.0 | 3.2 |

1. A vector for providing a cell of a certain genome with additional nucleic acid material integrated in its genome, whereby said vector comprises two transposase binding sites, whereby said transposase binding sites may be the same or different and are derived from a transposon found in another genus each in close proximity to a cut site for saiid transposase, whereby said additional nucleic acid material is located between said two transposase binding sites.

2. A vector according to claim 1, whereby the transposase binding sites and cut sites are based on the corresponding sites in transposons from the Tc1/mariner superfamily of transposons.

3. A vector according to claim 1 or 2, whereby the transposase binding sites and cut sites are based on Tc1-like transposons.

4. A vector according to claim 3, comprising at least the terminal 26 basepairs of Tc1 as a transposase binding site and a cut site for the Tc1 transposase.

5. A vector according to anyone of the aforegoing claims, further comprising a means for transducing a target cell.

6. A vector according to anyone of the aforegoing claims further comprising a nucleic acid sequence encoding transposase activity, functional for the transposase binding sites and cut sites present in said vector.

7. A vector according to anyone of the aforegoing claims, whereby the additional nucleic acid material encodes a protein.

8. A vector according to anyone of claims 1-7, whereby the additional nucleic acid material provides a blocking nucleic acid, such as an antisense RNA.

9. A vector according to anyone of the aforegoing claims, whereby said vector is derived from a viral vector, preferably an adenoviral, retroviral, or adeno associated viral vector.

10. A method for providing a target cell with additional nucleic acid material integrated into its genome comprising transducing said cell with a vector according to anyone of the aforegoing claims and providing said target cell with transposase activity functional for the transposase binding sites and cut sites present on said vector.

11. A method according to claim 10, whereby the transduction is carried out through infectious particles comprising the vector.

12. A recombined target cell obtainable by a method according to claim 10 or 11.

13. Use of a functional transposon found in a certain genus in integrating a nucleic acid of interest into the genome of a cell of a different genus.

14. Use according to claim 13, in a gene therapy regime.

15. Use according to claim 13 for producing transgenic animals.
